(19) United States
(12) Patent Application Publication
(10) Pub. No.: US 2005/0192321 A1
(43) Pub. Date: Sep. 1, 2005

(54) Treatment of neuropathy with rapid release aminopyridine

(76) Inventor: Jay M. Meythaler, Grosse Pointe Farms, MI (US)

Correspondence Address: GIFFORD, KRASS, GROH, et al., P.O. BOX 7021, TROY, MI, 48007-7021, US

(21) Appl. No.: 11/076,321

(22) Filed: Mar. 9, 2005

Related U.S. Application Data
(63) Continuation-in-part of application No. 10/885,175, filed on Jul. 6, 2004.
(60) Provisional application No. 60/502,991, filed on Sep. 15, 2003.
(60) Provisional application No. 60/513,750, filed on Oct. 23, 2003.

(51) Int. Cl. A61K 31/44

(52) U.S. Cl. 514/352

(57) ABSTRACT

A process for treating neuropathy includes administration to a patient suffering from a neuropathy more than 5 and less than 18 mg of aminopyridine in a dose. The aminopyridine is formulated in an immediate release formulation. At least two such doses are provided per day to create spike serum plasma levels of aminopyridine sufficient to drive the aminopyridine across the blood-nerve barrier.

### RELATED APPLICATIONS

This application is a continuation-in-part of U.S. patent application Ser. No. 10/885,175 filed Jul. 6, 2004, which claims priority of U.S. Provisional Patent Applications Ser. No. 60/502,991 filed Sep. 15, 2003 and Ser. No. 60/513,750 filed Oct. 23, 2003.

### BACKGROUND OF THE INVENTION

Multiple sclerosis is a degenerative and inflammatory neurological disease which affects the central nervous system. The mechanism of this disease is demyelinization of nerve fibers through degradation of the myelin sheath resulting in the short circuiting of nerve impulses with impeded transmission along nerve fibers. Symptoms associated with multiple sclerosis include spasticity, loss of motor strength, and painful dysesthesias. While myelin in the central nervous system is produced by oligodendrocites, there is no neurilemma, as exists in the peripheral nervous system. In the central nervous system myelin is not produced by Schwann cells and central nervous system nerve fibers are not capable of regeneration. Functionally, neurons are classified as afferent, efferent, or interneurons depending on the direction in which impulses travel relative to the central nervous system. Afferent neurons carry impulses from peripheral sense receptors to the central nervous system and are associated with long dendrites and relatively short axons. Conversely, efferent neurons transmit impulses from the central nervous system to effector organs such as muscles and glands and are characterized by short dendrites and long axons. Interneurons exist entirely within the central nervous system to form the linkage between afferent and efferent neurons and as such are characterized by short dendrites and axons of indeterminate comparative length. The peripheral nervous system is made up of nerves outside of the central nervous system and essentially divided into somatic and autonomic nervous systems. The peripheral nervous system includes cranial and spinal nerves, the somatic and autonomic systems, all of which are characterized by Schwann cell generated myelin sheaths.

In multiple sclerosis, axonic conduction fails during demyelination yet over time is restored in the surviving axons. The restoration of a demyelinated axon to functionality requires reducing the charge capacitance of the demyelinated axon. Remyelination of an axon associated with multiple sclerosis is characterized by short internodes and thin myelin plaque margins relative to normal myelin. A remyelinated axon readily becomes over stimulated through charge imbalance, recovery time between impulses is prolonged, and the axons that have been remyelinated become temperature sensitive.

Aminopyridines such as 4-aminopyridine (4-AP); 2,3-diaminopyridine (2,3-DAP); and 2,4-diaminopyridine (2,4-DAP) are known to be antagonists to muscle relaxants as detailed in U.S. Pat. No. 4,562,196. 4-aminopyridine has been shown to block potassium channels and improve conduction in neurons with damaged or destroyed myelin sheaths. While some multiple sclerosis patients report improvements in symptoms while taking 4-aminopyridine, the side effects associated with the treatment have generally outweighed the therapeutic benefits. The conventional wisdom is that multiple sclerosis therapy with 4-aminopyridine will improve by reducing side effects through the use of slow release 4-aminopyridine. (Schwid S R et al. Neurology 1997; 48(4):817-821). Unfortunately, 4-aminopyridine is highly toxic and slow release formulation trials have met with limited success.

Concerns about 4-AP side effects have led to studies of 2,3-DAP and 3,4-DAP to moderate the side effects and pharmacology of 4-AP. 2,4-DAP has been reported to induce less central nervous system stimulation than 4-AP with less toxicity than either 4-AP or 2,3-DAP.

Considerable development has gone into the development of drugs and delivery systems for the transport of pharmacologically active species across the blood-nerve barrier. Such attempts have included derivatizing a pharmacologically active species to include specific moieties recognized by various membrane receptors or alternatively to add lipophilic moieties. To date, while these approaches show promise, surprisingly little therapeutic progress has been made with respect to peripheral demyelinating disease.

Bioavailability enhancing additives known in the art to increase bioavailability, such as solubilizing agents, additives that increase drug permeability in the GI tract, and enzyme inhibitors are generally ineffective in moving compounds across the blood-brain or blood-nerve barriers. Likewise, solubilizing additives such as cyclodextrins and surfactants, as well as other additives that function to increase solubility like acidic or basic additives which solubilize a drug by changing the local pH in the GI tract to a pH where the drug solubility is greater than in the native system, are ineffective in moving compounds across the blood-brain or blood-nerve barriers.

An alternate approach to moving active compounds across the blood-nerve barrier has involved the use of long-acting drugs based on the premise a steady serum concentration is more likely to transport therapeutic amounts of active ingredient across the blood-brain barrier or blood-nerve barrier. However, clinical studies of long-acting 4-aminopyridine and tizanidine each individually demonstrated reduced central nervous system side effects, and the therapeutic efforts were likewise reduced as compared to short-acting versions of the same active species. As a result, one is forced to deliver high doses for therapeutic effect and to treat the resultant side effects with secondary compounds.

Thus, there exists a need for a method to deliver therapeutic quantities of mono- and diaminopyridines across the blood-nerve barrier in a manner that inhibits central nervous system side effects associated with such treatments.

### SUMMARY OF THE INVENTION

A process for treating neuropathy includes administration to a patient suffering from a neuropathy more than 5 and less than 18 mg of an aminopyridine in a dose. The aminopyridine is formulated in an immediate release formulation. At least two such doses are provided per day to create spike serum plasma levels of aminopyridine sufficient to drive the aminopyridine across the blood-nerve barrier.

### DETAILED DESCRIPTION OF THE INVENTION

The present invention has utility as a method for treating a demyelinating disease. An aminopyridine is defined to include monoaminopyridines such as 4-aminopyridine (4-AP); and diaminopyridines 2,3-diaminopyridine (2,3-DAP) and 2,4-diaminopyridine (2,4-DAP).

According to the present invention, an aminopyridine for the treatment of demyelinating disease, or a disease or injury involving neuronal damage, is delivered across the blood-nerve barrier through the administration of more than two doses daily of immediately active aminopyridine in dose amounts of greater than 5 milligrams (mg) and less than 18 mg per dose. It is appreciated that a dose of aminopyridine is a single aminopyridine or a mixture of two or three of the aminopyridines 4-AP, 2,3-DAP and 2,4-DAP. When a mixture is used preferably the mixture includes 4-AP and one of the diaminopyridine isomers present in a molar ratio of 5:1 to 1:3. Preferably, aminopyridine is delivered herein in three doses daily. More preferably, each dose includes 8 to 12 mg of aminopyridine per dose. The present invention utilizes a most preferred dose of 10 mg aminopyridine delivered three times daily to achieve therapeutic effect. Doses greater than 5 mg each of aminopyridine are able to create a blood spike concentration sufficient to force aminopyridine across the blood-nerve barrier. However, as doses increase beyond 12 mg of aminopyridine up to 18 mg of aminopyridine per dose, side effects associated with the treatment become more pronounced and are detrimental beyond 18 mg aminopyridine per dose. In the event that larger daily dosing is required, it is preferred that aminopyridine be given four or more times daily rather than increasing the quantity of aminopyridine delivered in a single dose. The aminopyridine is also optionally bonded to a dipeptide or larger peptide or a saccharide, and the added moiety is enzymatically cleaved once across the barrier in order to inhibit subsequent movement of the active therapeutic compound back across the barrier. Since the peak serum effect of a compound is hypothesized to be related to the quantity of the compound that is ultimately pushed across the blood-brain barrier or blood-nerve barrier, then by way of non-limiting theory, it is believed that a compound that reaches a peak serum level quicker therefore drives material across the blood-brain barrier or blood-nerve barrier to a greater extent.

According to the present invention it has been discovered that aminopyridine or modifying the same to create an intermediate HLB value of between 9 and 18 as determined by the methodology of Davies affords passage across the blood-nerve barrier (Davies J T in Proceedings of the International Congress on Surface Activity, 2^{nd} Ed. (Butterworth/Academic Press), 1957, p. 426), which is incorporated herein by reference. Moieties particularly well suited to modify the HLB value without adding additional charge to the aminopyridine include primary alkyl amines, secondary amines, tertiary amines, amides and oximes, where organic substituents on such moieties are selected for enzymatic cleavage to species that are native to nerve or brain tissues. Organic substituents operative herein illustratively include choline, choline derivatives, glucose, and lecithin components. Most preferably, the moiety is an amine or amide. Still more preferably, the aminopyridine according to the present invention is delivered contrary to the conventional wisdom as an immediate rapid release formulation so as to create a massive peak serum concentration. It has been discovered that a spiked dosage more efficiently moves the therapeutic compound across the barrier and thereafter compound that has crossed the barrier has a considerable half-life and compound that has remained in the circulatory system is readily cleared resulting in a higher effective dosage.

In addition to indications for multiple sclerosis, neuropathies that benefit therapeutically from the ability to deliver aminopyridine across the blood-nerve barrier include Guillain-Barre Syndrome (GBS); chronic demyelinating polyradiculoneuropathy (CIDP), diabetic mellitus; and the hereditary sensory-motor neuropathies such as Charcot-Marie-Tooth disease, Friedrich's ataxia, Lambert-Eaton Myasthenic Syndrome, porphyria, lipoprotein neuropathies, and familial amyloid neuropathies.

Contrary to the prior art where sustained release or immediate release oral doses of aminopyridine are given daily, the present invention treats demyelinating diseases through the administration by oral or parenteral routes of fast-acting aminopyridine. It is appreciated that other routes of administration are also operative herein, these alternate routes illustratively including intracisternally, intrathecally, intravaginally, intraperitoneally, intravesically, or as a buccal or nasal spray. Preferably, a compound therapeutically active against peripheral demyelinating disease is delivered parentally. More preferably, the therapeutic compound is delivered by intravenous parenteral injection.

Aminopyridine doses are typically greater than 10 and less than 80 mg and are delivered twice daily, three times daily, or more frequently. Regardless of the mode of administration, whether by immediate release oral capsule or intravenous parenteral injection, a massive peak serum concentration of a therapeutic compound serves to drive a therapeutic compound across the blood-nerve barrier. Preferably, intravenous injection is by push. Once an aminopyridine has crossed the physiological barrier, rapid transported through cell membranes follows and 4-AP thereafter has a considerable pharmacological half-life. This has been demonstrated in 4-aminopyridine therapies. Segal J et al. Pharmacotherapy 1999; 19:713-23, which is incorporated herein by reference.

Compositions suitable for parenteral injection optionally include physiologically acceptable sterile aqueous or non-aqueous solutions, dispersions, suspensions or emulsions. Examples of suitable aqueous and non-aqueous carriers, diluents, solvents and vehicles illustratively include water; ethanol; polyols, such as propylene glycol, polyethylene glycol, glycerol, and the like; combinations thereof; and injectable organic esters, such as ethyl oleate.

Therapeutic compositions optionally also include adjuvants such as preservatives, wetting agents and emulsifiers. Prevention of the action of microorganisms is assured through the addition of various antibacterial and antifungal agents, illustratively including parabens, chlorobutanol, phenol, sorbic acid, and the like. Isotonic agents are also optionally operative herein and illustratively include sugars, sodium chloride and the like.

Solid dosage forms for oral administration include capsules, tablets, pills, powders, and granules. In solid dosage forms, the therapeutic compound is admixed with at least one inert customary excipient illustratively including sodium citrate or dicalcium phosphate, or a filler, illustratively including a starch, lactose, sucrose, glucose, mannitol and silicic acid. Additionally, a binder, humectant, disintegrating agent, solution retarder, absorption accelerator, wetting agent, absorbent or lubricant is operative herein as detailed in U.S. Pat. No. 6,503,931 which is incorporated herein by reference. An oral dosage according to the present invention is formed by mixing reagent grade or better 4-aminopyridine that contains minimal quantities of isomers or contaminants that can interfere with barrier transport and a mixing agent. Mixing agents operative herein are chemically and biologically inert and illustratively include: cellulose acetate phthalate; cellulose acetate trimaletate; hydroxy propyl methylcellulose phthalate; polyvinyl acetate phthalate; ammonio methacrylate copolymers such as those sold under the Trade Mark EUDRAGIT® RS and RL; poly acrylic acid and poly acrylate and methacrylate copolymers such as those sold under the Trade Mark EUDRAGIT® S and L; polyvinyl acetaldiethylamino acetate; hydroxypropyl methylcellulose acetate succinate; shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch; and cellulose based cross-linked polymers such as hydoxypropyl cellulose, hydroxypropyl methylcellulose, polyvinylpyrrolidone, crosslinked starch, microcrystalline cellulose, chitin; aminoacryl-methacrylate copolymer (EUDRAGIT® RS-PM, Rohm & Haas); pullulan; collagen; casein; agar; gum arabic; sodium carboxymethyl cellulose; (swellable hydrophilic polymers) poly(hydroxyalkyl methacrylate) (m. wt. about 0.5 k-5,000 k); polyvinylpyrrolidone (m. wt. about 0.10 k-360 k); anionic and cationic hydrogels; copolymers of maleic anhydride and styrene, ethylene, propylene or isobutylene, pectin; polysaccharides such as agar, acacia, karaya, tragacanth, algins and guar; polyacrylamides; POLYOX® polyethylene oxides, diesters of polyglucan, crosslinked polyvinyl alcohol and poly N-vinyl-2-pyrrolidone, sodium starch glucolate; hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides, methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid, sorbitan esters, natural gums, lecithins, pectin, alginates, ammonia alginate, sodium, calcium, potassium alginates, propylene glycol alginate, agar; and gums such as arabic, karaya, locust bean, tragacanth, carrageens, guar, xanthan, scleroglucan; and mixtures, and blends of the aforementioned mixing agents.

The resulting mixture is loaded into a fast dissolution conventional hard gelatin capsule of size 1 or 2, to assure a burst release of the aminopyridine. Typically, solid aminopyridine is ground by ball milling into uniform granules. The resulting aminopyridine granules are then uniformly mixed with one of the above mixing agents such as powdered methyl cellulose where the aminopyridine constitutes from 0.5 to 10% by weight aminopyridine and preferably from 1.5 to 4.5% by weight aminopyridine. The filler dispersed aminopyridine is then pressed into tablets, packed into a capsule or other conventional solid oral medicament packing as detailed in Remington's Pharmaceutical Sciences, 16th Ed., 1980, Mack Publishing Co., Easton, Pa.

In the preferred embodiment, the therapeutic compound is administered independent of various adjuvants which potentially interfere with the largely unknown mechanism by which aminopyridine crosses the blood-nerve barrier.

According to the present invention, a large peak serum level of aminopyridine allows a considerable quantity of the aminopyridine to cross the blood-nerve barrier and with a quickly declining serum level of the aminopyridine through barrier crossing and physiological clearing of the aminopyridine, considerably fewer side effects are noted while delivering an efficacious dose. Aminopyridine has an active site half-life of an excess of two days. This half-life allows for continued therapeutic effect even in the event of missed doses.

It is appreciated that in addition to an active therapeutic compound, a pharmaceutically acceptable salt, ester, amide or prodrug thereof is also readily administered.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, Barge S M et al. Pharmaceutical Salts. J. Pharm. Sci., 1977; 66:1-19 which is incorporated herein by reference.) Examples of pharmaceutically acceptable, non-toxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods.

Examples of pharmaceutically acceptable, non-toxic amides of the compounds of this invention include amides derived from ammonia, primary C₁-C₆ alkyl amines and secondary C₁-C₆ dialkyl amines wherein the alkyl groups are straight or branched chain. In the case of secondary amines, the amine may also be in the form of a 5- or 6-membered heterocycle containing one nitrogen atom. Amides derived from ammonia, C₁-C₃ alkyl primary amines, and C₁-C₂ dialkyl secondary amines are preferred. Amides of the compounds of the invention may be prepared according to conventional methods.

The term “prodrug” refers to compounds that are rapidly transformed in vivo to yield the parent compounds of the above formula, for example, by hydrolysis in blood. A thorough discussion is provided in Higuchi T and Stella V, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.

In addition, an aminopyridine can exist in unsolvated as well as solvated forms with pharmaceutically acceptable solvents such as water, ethanol, and the like. In general, the solvated forms are considered equivalent to the unsolvated forms for the purposes of the present invention.

The present invention is further illustrated by the following clinical examples. These examples are not intended to limit the scope of the specification, including the claims, in any way. While the following trial data used as a participant selection criteria symptoms of Guillain-Barre Syndrome, cross sensitivity between the Miller-Fisher Syndrome variant of Guillain-Barre Syndrome and multiple sclerosis is well known to the art (Ropper A H. The Guillain-Barre Syndrome. N. Eng. J. Med. 1992; 326:1130-1136; Meythaler J M. Rehabilitation of Guillain-Barre Syndrome: A Review. Arch. Phys. Med. Rehabil. 1997; 78:872-9; Ashbury A K et al. Criteria for Diagnosis of Guillain-Barre Syndrome. Ann. Neurol. 1978; 3:565-566; Goust J M et al. Abnormal T cell subpopulations in circulating immune complexes in the Guillain-Barre syndrome and multiple sclerosis. Neurology 1978 28(5):421-5).

### EXPERIMENTAL DATA

Study Design

This is a Phase II double-blind, placebo controlled, crossover, dose-escalating study in subjects with MS and SCI. An initial trial of ten patients is randomized to one of the two treatment sequences (A or B) as shown below.

Population

The study population consists of subjects with GBS injury whose neurological status has been stable for at least eighteen months. Ten subjects are enrolled.

### Inclusion Criteria

Male or female, 18 to 75 years of age, irrespective of race.
The subject is able to and has voluntarily given informed consent prior to the performance of any study specific procedures.
The subject has neurological impairment secondary to GBS which has been stable for at least twelve months.
The subject has motor strength averaging between 3.0 and 5.0 on the ASIA motor scale.
The subject is able and willing to comply with the protocol.

### Exclusion Criteria

The subject is a pregnant female (as determined by a urine pregnancy test), a lactating female, or a female of child-bearing potential not using one of the following methods of birth control (oral contraceptive, implantable conception device or injectable contraceptive agent, barrier method of contraception) or not surgically sterilized.
The subject has a history of seizures.
The subject has a known allergy to pyridine-containing substances.
The subject has evidence of upper motor neuron involvement.
The subject has any medical condition, including psychiatric disease, which would interfere with the interpretation of the study monitor.
The subject has been on concomitant medications at a stable dose/regimen for less than three weeks, and/or the stable dose/regimen of concomitant medications is expected to be changed during the course of the study.
The subject has a history of drug or alcohol abuse within the past year.
The subject has received an investigational drug within thirty days prior to the screening visit.
The subject has taken 4-aminopyridine in the past, whether through participation in a previous study or self-medication.
   Objective Neurological Functional Assessment

Variables to be Collected: The measures of neurological status reported for this study are:

Motor strength was rated on the traditional 0-5 ordinal scale:
0—absent motor strength
1—trace motor strength
2—can move the specified joint but only with gravity eliminated
3—can move the joint against gravity but not against any opposing force
4—can move the joint against opposing force but the strength is not normal for the person or symmetrical
5—normal motor strength

This scale is employed to measure the following motor strength for each of these joint motions on both the right and the left sides: hip flexion, hip adduction, hip abduction, knee flexion, knee extension, ankle dorsiflexion, ankle plantar flexion, shoulder abduction, elbow extension, elbow flexion, wrist flexion, and wrist extension.

In addition, the following criteria are also rated:

Hand grip strength is measured on a hand dynamometer that had been calibrated. Each patient is given three trials separated by thirty-second rest periods and the strongest of the three measurements is recorded for each hand.

In addition, serum laboratories are drawn at the beginning of the study and every week of the study. The serum laboratories included glucose, blood urea nitrogen, creatinine, uric acid, calcium, total protein, albumin, phosphate, total bilirubin, cholesterol, LDH, SGOT/AST, alkaline phosphatase, hematocrit, hemoglobin, red blood cell count, platelet count, and white blood cell count with differential.

Treatment variables to be collected include method of bladder management as well as usage of a ventilator, plasmapheresis, steroids, and intravenous immunoglobulin (IVIg) as these variables are related to the severity of disease (Zelig G et al. The rehabilitation of patients with severe Guillain-Barre syndrome. Paraplegia 1988; 26:250-254; Meythaler J M et al. Rehabilitation outcomes of patients who have developed Guillain-Barre Syndrome. Am. J. Phys. Med. Rehabil. 1997; 76:411-419). Information will also be collected on GBS subtype, GBS etiology, general patient demographic characteristics, relevant medical history, length of stay during acute care and rehabilitation, charges for acute care and rehabilitation, sponsors of care, and rehospitalizations (Meythaler J M. Rehabilitation of Guillain-Barre Syndrome. Arch. Phys. Med. Rehabil. 1997; 78:872-9; Meythaler J M et al. Rehabilitation outcomes of patients who have developed Guillain-Barre syndrome. Am. J. Phys. Med. Rehabil. 1997; 76:411-9).

Data Analysis/Database Development

The collectibility and ultimate quality of information contained in each variable is assessed both subjectively and objectively.

The entire project team reviews the reported frequency distributions, means, cross-tabulations, etc. of each variable for reasonableness. For example, if a high or low incidence of a particular complication is reported that seems inconsistent with the clinical experience of the investigators, then the definition of this variable is reconsidered and either left unchanged, clarified or deleted as appropriate. Complications that do not occur in any cases are also candidates for deletion, while unanticipated occurrences may be candidates for addition to the database. This procedure has been used successfully by the Model Spinal Cord Injury Systems for many years.

### EMG NCS

All patients will have two upper and lower extremity motor and sensory nerve conditions (total four motor four sensory) performed at the enrollment period and at the maximal point of drug delivery in both the A and B phases. This assesses for objective improvement in nerve conduction velocity with the use of 4-AP. Nerve conduction velocities and amplitudes are performed for median and peroneal nerves.

Dosing sequence

4-AP is compounded in a #1 quick dissolution capsule containing 5 mg 4-AP and 235 mg lactose. Subjects are randomized to a double-blind, placebo-controlled, cross-over design, which has two eight-week treatment arms with a three-week washout. The average dosage at four weeks is a fast release 4-AP delivered three times daily with 10 mg 4-AP per dose for a total of 30 mg per day. Patients who demonstrate improvement are continued on the mediation at this level for an additional three months. Assessments are performed every two weeks during the randomized trial and every month for those continued for up to three months on the medication.

Results

Ten patients were recruited for the double-blind trial.

Motor strength Lower extremity strength for hip abduction, hip adduction, hip flexion, knee flexion, ankle dorsiflexion and plantar flexion increased 15% on average in patients taking 4-AP relative to the placebo.

Upper extremity strength for hip abduction, hip adduction, hip flexion, knee flexion, ankle dorsiflexion and plantar flexion increased 15% on average in patients taking 4-AP relative to the placebo.

In view of the teaching presented herein, other modifications and variations of the present inventions will be readily apparent to those of skill in the art. The discussion and description are illustrative of some embodiments of the present invention, but are not meant to be limitations on the practice thereof. It is the following claims, including all equivalents, which define the scope of the invention.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

1. A process for treating neuropathy comprising administering to a patient suffering from neuropathy more than 5 and less than 18 mg of an aminopyridine selected from the group consisting of: 4-aminopyridine, 2,3-diaminopyridine, 2,4-diaminopyridine, and combinations thereof per dose in an immediate release formulation in at least two doses per day.

2. The process of claim 1 wherein said aminopyridine is delivered in three doses per day.

3. The process of claim 1 wherein said aminopyridine is delivered from 8 to 12 mg per dose.

4. The process of claim 2 wherein said aminopyridine is delivered from 8 to 12 mg per dose.

5. The process of claim 1 wherein said aminopyridine is delivered in conjunction with physiologically acceptable sterile aqueous or non-aqueous material selected from the group consisting of: a carrier, a diluent, a solvent and a vehicle.

6. The process of claim 1 wherein the administration is independent of a sustained release version of aminopyridine.

7. The process of claim 1 wherein administration is parenterally.

8. The process of claim 7 wherein administration is by intravenous parenteral injection.

9. The process of claim 8 wherein said intravenous parenteral injection is an intravenous push injection.

10. The process of claim 1 wherein said aminopyridine is delivered in total at from 10 to 80 mg per day.

11. The process of claim 1 wherein administration is orally.

12. The process of claim 11 wherein administration is by way of a solid dosage form.

13. A process for treating neuropathy comprising the step of: administering orally to a patient suffering neuropathy between 8 and 12 mg of an aminopyridine selected from the group consisting of: 4-aminopyridine, 2,3-diaminopyridine, 2,4-diaminopyridine, and combinations thereof in a dose in an immediate release formulation in three doses per day.

14. The process of claim 13 wherein said aminopyridine is delivered in conjunction with physiologically acceptable sterile aqueous or non-aqueous material selected from the group consisting of: a carrier, a diluent, a solvent and a vehicle.

15. The process of claim 13 wherein the administration is independent of a sustained release version of said aminopyridine.

16. A process for manufacturing an immediate release version of an aminopyridine comprising mixing a granulated aminopyridine selected from the group consisting of: 4-aminopyridine, 2,3-diaminopyridine, 2,4-diaminopyridine, and combinations thereof with a granulated inert mixing agent to form a mixture where aminopyridine constitutes from 1.5 to 4.5% by weight; and
filling a capsule with said mixture.

17. The process of claim 16 wherein said aminopyridine is delivered in conjunction with physiologically acceptable sterile aqueous or non-aqueous material selected from the group consisting of: a carrier, a diluent, a solvent and an inert vehicle.

18. A capsule produced according to the process of claim 16.
